(19) United States
(12) Patent Application Publication
(10) Pub. No.: US 2014/0338667 A1
(43) Pub. Date: Nov. 20, 2014

(54) Bellows Bulb Occlusion Valve

(71) Applicant: Parker-Hannifin Corporation, Cleveland, OH (US)

(72) Inventors: Paul S. Baczkowski, Orchard Park, NY (US); Seth Wolf, Fort Washington, PA (US)

(73) Assignee: PARKER-HANNIFIN CORPORATION, Cleveland, OH (US)

(21) Appl. No.: 14/277,827

(22) Filed: May 15, 2014

Related U.S. Application Data
(60) Provisional application No. 61/823,418, filed on May 15, 2013.

(51) Int. Cl. A61M 16/20

(52) U.S. Cl. CPC A61M 16/201
USPC 128/203.28

(57) ABSTRACT

A flow control device for blocking the flow of analgesic gas to the mask of an analgesic system includes a bellows-bulb at the distal end of the breathing circuit. The device can be easily operated by hand. By depressing the bellows when the mask is removed, gaseous pollution, such as nitrous oxide or other gas pollution, can be controlled while the system is purged.

### CROSS REFERENCE TO RELATED APPLICATIONS

This patent application claims the benefit of the filing date of U.S. Provisional Patent Application Ser. No. 61/823,418 filed May 15, 2013, the disclosure of which is incorporated herein by reference in its entirety.

### TECHNICAL FIELD

The present invention relates to a flow control device, and more particularly to a valve, for example, as may be used in analgesia breathing system.

### BACKGROUND

Gaseous analgesia, such as nitrous oxide/oxygen, can be delivered to a patient via a continuous flow system or a demand flow system. In a continuous flow system sedation system, analgesic gas flow is established at the mixing head and is delivered to a patient end of a breathing circuit via tubing, directional control valves, and a face mask. If the face mask is maintained on the patient's face, the gas flows through a single tube, through the face mask and into the patient's lungs. Exhaled gas from the lungs is conducted through the mask and through a second tube to scavenging equipment. The operation is similar with a breathing circuit connected to a demand flow device, except that in a demand flow device, the patient's inhalation initiates the gas flow.

In some systems, a full face mask is used to deliver analgesia to a patient. In such systems, it can be desirable to use a disposable breathing circuit, such as a co-axial circuit. A shortcoming of a co-axial circuit relates to the ability of the circuit to dump excess analgesic gas into the administration room when the mask is removed from the patients face, thereby creating a pollution hazard for physicians and staff. The potential for pollution is well documented and recommended limits have been established to control occupational exposure by physicians and staff.

Pollution can occur any time the mask is removed from the patient's face. This can be an obscure but important source of treatment room pollution. For example, mixed gas (such as nitrous oxide) may continue to flow when the mask is removed from the patient's face. This gas is not taken into the patient's lungs or through the exhalation tubing to the scavenging equipment. Pollution also can occur any time the mask-to-patient face seal is abrogated.

Many safety conscious biomedical departments regularly check for pollution sources, finding even extremely minute leaks in gas mixing heads. While it is common to scavenge gas exhaled from a patient during a medical procedure, there often is little control over the analgesia pollution that results from actions of the physicians or staff during the procedure.

### SUMMARY

The present invention includes a flow control device for blocking the flow of analgesic gas to the mask of an analgesic system. The flow control device (also referred to herein as a valve) includes bellows-bulb at the distal end of the breathing circuit that can be easily operated by hand. By depressing the bellows when the mask is removed, gaseous pollution, such as nitrous oxide or other gas pollution, can be controlled while the system is purged. The device affords the physician or administering staff a simple mechanism for reducing or eliminating analgesic gas pollution. Once the operator seals the end of the delivery tubing and removes the mask from the patient's face, all mixed gas is conducted to the scavenging system and pollution to the surrounding environment can be reduced or eliminated.

Another advantage is that the flow control device can block mixed gas from flowing to the patient while a physician or administering staff adjust the settings on the breathing circuit. For example, the flow control device can block the flow of mixed gas to the patient and redirect the mixed gas towards the scavenging system of the breathing circuit. The breathing circuit can then be purged, e.g., with pure oxygen, prior to removing the mask from the patient. As a result, mixed gas can be turned off and the breathing circuit safely purged with minimal or no gas pollution.

Additionally, the presence of the device on a breathing circuit can encourage the physician or administering staff to be in control and mindful of the rogue pollution source of which they may not have been previously aware.

The device also allows the administering personnel full control of a pollution source. The device can be used at any time during a procedure to safely and completely block the flow of mixed gases to the mask. For example, the device can be used in middle of a procedure to allow the patient to remove the mask to speak to the physician or other staff as may be necessary or desired during the procedure.

According to one exemplary embodiment, a valve for an analgesia breathing system includes a body having a passageway through which gas flows and an occluding element having an inflatable bulb disposed in the passageway and a compressible actuation portion disposed external to the passageway. The compressible actuation portion being compressible to inflate the bulb to seal the passageway, thereby inhibiting gas flow though the passageway.

According to another exemplary embodiment, a valve for an analgesia breathing system includes a body having a first opening and a second opening in fluid communication with one another via a passageway and an occluding element. The occluding element has an inflatable bulb disposed in the passageway and a bellows member disposed external to the passageway, the bellows member being compressible to inflate the bulb to occlude the passageway.

According to another exemplary embodiment, a hollow occluding device for a breathing circuit includes an inflatable bulb and a compressible actuator. When the compressible actuator is compressed, fluid in the occluding device shifts from the actuator to the bulb and causes the bulb to inflate, and when the actuator is uncompressed, fluid in the occluding device shifts back to the actuator and the bulb deflates.

These and further features of the present invention will be apparent with reference to the following description and attached drawings. In the description and drawings, particular embodiments of the invention have been disclosed in detail as being indicative of some of the ways in which the principles of the invention may be employed, but it is understood that the invention is not limited correspondingly in scope. Rather, the invention includes all changes, modifications and equivalents coming within the spirit and terms of the claims appended hereto. Features that are described and/or illustrated with respect to one embodiment may be used in the same way or in a similar way in one or more other embodiments and/or in combination with or instead of the features of the other embodiments.

### BRIEF DESCRIPTION OF THE DRAWINGS

Embodiments of this invention will now be described in further detail with reference to the accompanying drawings, in which:

FIG. 1 is a schematic block diagram of an analgesia breathing system.

FIG. 2 is a cross-sectional view of flow control device in the open position; and

FIG. 3 is a cross-sectional view of flow control device in the closed position.

### DETAILED DESCRIPTION

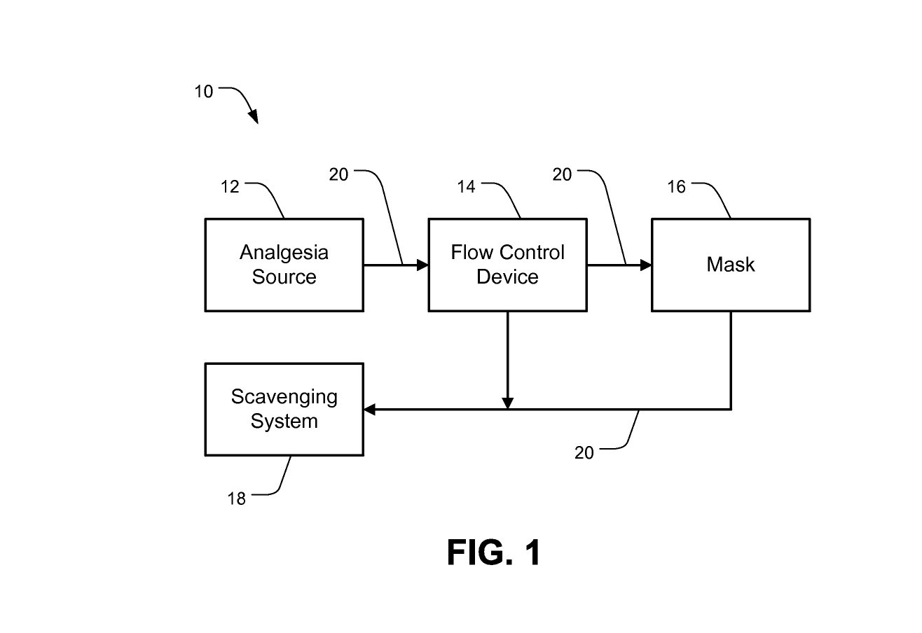

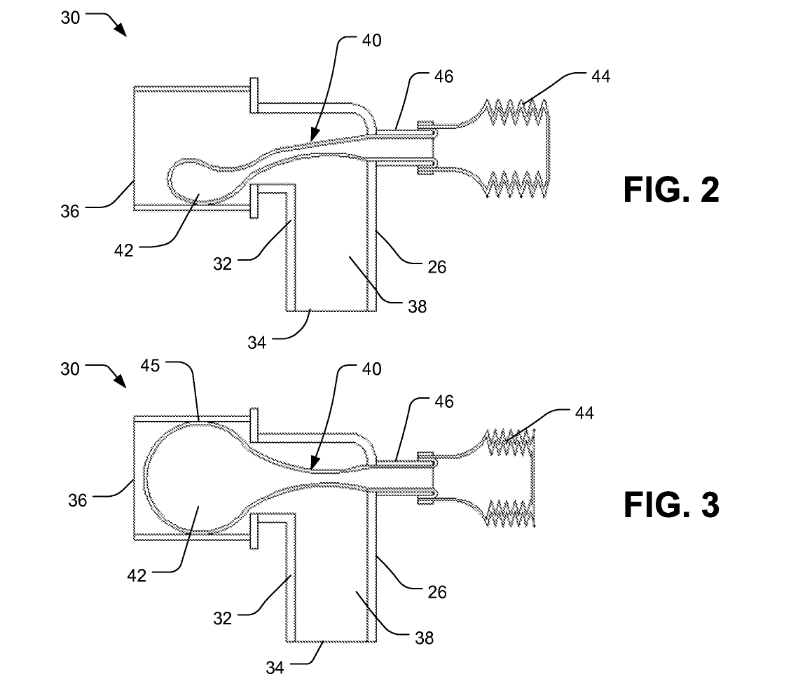

Referring initially to FIG. 1, an exemplary analgesic gas system 10 is shown in schematic form. The system can include an analgesia source 12 such as a mixed gas, for example, nitrous oxide. The gas can flow through a flow control device 14, such as the valve shown in FIGS. 2 and 3. The flow control device can be operated control whether the gas flows to a mask 16, where it is administered to a patient, or the gas can be diverted to a scavenging system 18. The various components of the system can be coupled to one another with tubing, shown generally with reference numeral 20.

The flow control device 14 may be located proximate to the mask of the analgesic system. By locating the flow control device near the patient, it is possible to contain the analgesic gas in the system rather than allowing the gas to pollute the surrounding environment. Stated another way, occluding the breathing circuit at the end closest to the patient outlet contains the gas already inside the breathing circuit tubing, and allows the operator of the equipment time to stop mixed gas flow at the gas mixing device and purge the entire breathing circuit following standard operating procedures. This can reduce or eliminate the amount of analgesic gas that may pollute the surrounding environment.

FIGS. 2 and 3 show an exemplary embodiment of the flow control device of the system of FIG. 1. In FIGS. 2 and 3, the flow control device is shown as a valve 30. The valve 30 is formed from a body 32 that has a first opening 34 configured to be coupled to tubing that supplies gas from an analgesic source and a second opening 36 configured to be coupled to tubing that supplies the gas to a mask. The first opening and the second opening are in fluid communication with one another via a passageway 38 through the valve.

At least partially disposed in the passageway 38 is an occluding element 40. The occluding element includes an inflatable bulb 42 and a actuation portion 44. As shown in FIGS. 2 and 3, the inflatable bulb 42 is primarily disposed in the passageway of the valve body and the actuation portion 44 is disposed external to the passageway outside of the valve body. The occluding element may be sealed, for example, hermetically sealed or may be mechanically captured and sealed. The occluding element may be substantially hollow and may contain a fluid, such as air, that is easily shifted back and forth from the actuation portion to the bulb.

The inflatable bulb 42 may be shaped similar to a small balloon, and may have an elasticity that allows it to inflate as required, and occlude the flow of gas. The valve may be formed from any low modulus, latex material or non-latex material such as silicone or thermoplastic elastomer (TPE).

The actuation portion 44 may be compressible. In one embodiment, the actuation portion 44 is resiliently deformable such that it can be compressed by application of a compression force (for example, an axial compression force) but return to its natural shape when the compression force is removed. The actuation member may be a bellows member (for example, a convoluted pipette bellows), which can be compressed through application of an axial compression force. Axial compression of the actuation portion inflates the bulb to seal the passageway, and removal of the compression force from the actuation portion deflates the bulb to open the passageway. The actuation portion may be hand actuated, for example, by the thumb of an operator.

The bulb 42 and actuation portion 44 are functionally coupled to one another such that compression of the actuation portion imparts a corresponding inflation of the bulb. When the actuation portion is in its relaxed (uncompressed) state shown in FIG. 2, the majority of air in the occluding element is located in the actuation portion while the bulb contains little or no air. Actuation of the actuation portion shifts air inside of the occluding element from the actuation portion to bulb, causing the bulb to inflate and radially expand to engage against an internal surface of the 45 passageway as shown in FIG. 3. In this position, the passageway is occluded or sealed and the flow of gas through the valve is blocked, thereby inhibiting or preventing the analgesic gas from escaping into the treatment room. When the compressive force on actuation portion is released, its natural spring action returns it to its initial shape and removes the air that was contained in the bulb causing the bulb to deflate, thereby opening the passageway as shown in FIG. 1 to allow gas to through the valve to the mask.

The occluding element 40 may be assembled onto the valve body 32 as shown in FIGS. 2 and 3. For example, the valve body 11 can include projection 46 that opens into the passageway 38. The bulb can be inserted into the passageway through the projection 46. The bulb can be secured to the body by wrapping a portion 48 of the bulb around the projection 46. The actuation portion 44 can then be slid over the wrapped portion 48 and a securing element, such as a band or lock ring, can be applied to couple both element securely to the projection. The assembly of the bulb and actuation portion to the body in this manner forms a hermetically sealed interior space of the occluding member, which can be actuated to occlude the passageway as described herein.

If used whenever the mask is not on the patient's face, the flow control device as disclosed herein can substantially reduce or prevent potentially harmful trace analgesia gas from entering the treatment room.

The concepts described herein can be implemented in various devices, but is primarily shown and described as contained in the elbow that attaches the face mask to the analgesia breathing circuit. Although the principles, embodiments and operation of the present invention have been described in detail herein, this is not to be construed as being limited to the particular illustrative forms disclosed. They will thus become apparent to those skilled in the art that various modifications of the embodiments herein can be made without departing from the spirit or scope of the invention.

1. A valve for an analgesia breathing system comprising:
a body having a passageway through which gas flows;
an occluding element having an inflatable bulb disposed in the passageway and a compressible actuation portion disposed external to the passageway, the compressible actuation portion being compressible to inflate the bulb to seal the passageway, thereby inhibiting gas flow though the passageway.

2. The valve of claim 1, wherein the actuation portion includes a bellows member.

3. The valve of claim 2, wherein the bellows member is axially compressible to inflate the bulb.

4. The valve of claim 3, wherein the bellows member is resiliently deformable.

5. The valve of claim 5, wherein application of an axial compression force to the bellows member causes the bulb to inflate and removal of the axial compression force causes the bulb to deflate.

6. The valve of claim 5, wherein the occluding element is hermetically sealed.

7. The valve of claim 1, wherein inflation of the bulb effects a radial expansion of the bulb against an internal surface of the passageway.

8. A valve for an analgesia breathing system comprising:
a body having a first opening and a second opening in fluid communication with one another via a passageway;
an occluding element having an inflatable bulb disposed in the passageway and a bellows member disposed external to the passageway, the bellows member being compressible to inflate the bulb to occlude the passageway.

9. The valve of claim 8, wherein the occluding element is hollow.

10. The valve of claim 9, wherein the occluding element is hermetically sealed.

11. The valve of claim 10, wherein inflation of the bulb effects a radial expansion of the bulb against an internal surface of the passageway.

12. The valve of claim 11, wherein the bellows member is axially compressible to inflate the bulb.

13. The valve of claim 12, wherein the bellows member is resiliently deformable.

14. The valve of claim 13, wherein application of an axial compression force to the bellows member causes the bulb to inflate and removal of the axial compression force causes the bulb to deflate.

15. A hollow occluding device for a breathing circuit comprising an inflatable bulb and a compressible actuator, wherein when the compressible actuator is compressed, fluid in the occluding device shifts from the actuator to the bulb and causes the bulb to inflate, and when the actuator is uncompressed, fluid in the occluding device shifts back to the actuator and the bulb deflates.

16. The occluding device of claim 15, wherein the occluding device is hermetically sealed.

17. The occluding device of claim 15, wherein inflation of the bulb effects a radial expansion of the bulb.

18. The occluding device of claim 15, wherein the compressible actuator includes a bellows member that is axially compressible to inflate the bulb.

19. The occluding device of claim 18, wherein application of an axial compression force to the bellows member causes the bulb to inflate and removal of the axial compression force causes the bulb to deflate.

20. The occluding device of claim 19 in combination with a valve body, wherein the inflatable bulb of the occluding device is disposed internal to the valve body and the compressible actuator is disposed external to the valve body, such that when the inflatable bulb is inflated, it seals against the tube, thereby inhibiting a flow of gas through the valve body.
